(19) United States
(12) Patent Application Publication
(10) Pub. No.: US 2007/0111241 A1
(43) Pub. Date: May 17, 2007

(54) System and method for accessing, tracking, and editing sequence analysis and software to accomplish the same

(76) Inventors: Nezih Cereb, Ossining, NY (US); Soo Young Lee, Ossining, NY (US)

Correspondence Address: GREENBERG TRAURIG, LLP, MET LIFE BUILDING, 200 PARK AVENUE, NEW YORK, NY, 10166, US

(21) Appl. No.: 11/582,008

(22) Filed: Oct. 16, 2006

Related U.S. Application Data
(60) Provisional application No. 60/727,011, filed on Oct. 14, 2005.

(51) Int. Cl. C12Q 1/68; G06F 19/00

(52) U.S. Cl. 435/6; 702/20

(57) ABSTRACT

The present invention relates to a system and method for accessing, tracking and editing sequence analysis and software to accomplish the same. The present invention includes embodiments that permit a party, for example a customer, to track the status of the samples (e.g., tissue, blood, or DNA) that the party sends for sequence based typing (“SBT”) analysis. The party can also participate in the analysis by accessing the sequencing data for the submitted samples. A party is able to remotely access tools for sequence based typing (e.g., Histomatcher), and using such tools, the party can review and edit the data. The party can also generate reports via the accessed tools.

This application claims priority from provisional application 60/717,011 filed Oct. 14, 2005, which is incorporated herein by reference in its entirety.

### INTRODUCTION

The present invention relates to a network based system and method permitting a party to access and interact with inventions directed toward sequence analysis and software to accomplish the same.

### BACKGROUND OF THE INVENTION

The inventions directed toward sequence analysis and software to accomplish the same are described in U.S. Provisional Application 60/662,738, the entirety of which is incorporated herein. The incorporated invention in a first embodiment, called HistoMatcher™, is a web based tool developed for Sequence Based typing analysis. A second embodiment, called Histotie™, is designed to integrate two types of experiments and results, such as DNA hybridization and DNA sequencing. A third embodiment, called Histotype™, is designed for Sample Tracking, Data handling, SSOP Typing Analysis and Database Management.

### SUMMARY OF THE INVENTION

The present invention includes embodiments that permit a party, for example a customer, to track the status of the samples (e.g., tissue, blood, or DNA) that the party sends for sequence based typing (“SBT”) analysis. The party can also participate in the analysis by accessing the sequencing data for the submitted samples. A party is able to remotely access tools for sequence based typing (e.g., Histomatcher), and using such tools, the party can review and edit the data. The party can also generate reports via the accessed tools. A description of the tools and their implementation, including as can be incorporated into the present invention, is found in Exhibit 1 to the priority application 60/727,011, the entirety of which is incorporated herein. A schematic drawing of a network based system is shown at FIG. 2, which shows parties 110a, 110b connected to a SBT analysis service 110 (e.g., Histogentics). As used herein, the SBT analysis service refers to the service provider as well as the services, technology, and software provided by provider, as description of which can be found in the present application and the incorporated references, including Exhibit 1.

### DESCRIPTION OF THE DRAWINGS

FIG. 1 is an exemplary embodiment of the Outsource HLA-SBT/Outsource SBT system and method.

FIG. 2 is schematic of a network based system.

FIG. 3 relates to the Histomatcher™. FIG. 3A shows the automatic arrangement of sequencing raw data files. FIG. 3B shows automatic project creating and counting calculation. FIG. 3C shows the SBT analysis main screen. FIG. 3D shows the mutation review. FIG. 3E shows the Histomatcher™ main screen in detail.

FIG. 4 also relates to the Histomatcher™. FIG. 4A shows how to edit a mutation. FIG. 4B shows how to conform a mutation. FIG. 4C shows the reporting.

FIG. 5 relates to the Histotie. FIG. 5A shows the main window. FIG. 5B shows the edit mode screen. FIG. 5C shows the chromatograms view. FIG. 5D shows the probe reaction view.

FIG. 6 relates to Histoype. FIG. 6A shows the typing request from NMDP by email. FIG. 6B shows importing the typing request. FIG. 6C shows grouping and automatic OS creation. FIG. 6D shows a sample 12×8 orientation sheet. FIG. 6E shows filter paper punching script generation. FIG. 6F shows filter paper punching.

FIG. 7 also relates to Histotype. FIGS. 7A and 7B show score input. FIG. 7A shows a probe reaction film after hybridization and FIG. 7B shows blotting arrangement. FIG. 7C shows a probe reaction scores. FIG. 7D shows importing probe reaction score into the Histotype. FIG. 7E shows applying threshold % range to find +ve, −ve reactions. FIG. 7F shows a review of converted probe scores. FIG. 7G shows an analysis. FIG. 7H shows a sample probe bit pattern in Excel. FIG. 7I shows a pattern chart. FIG. 7J shows ambiguous combo detection. FIG. 7K shows cherry picking the ambiguous samples. FIG. 7L shows adding sequencing primers. FIG. 7M shows sequence based typing analysis entry.

FIG. 7N shows combining SSOP and SBT results.

FIG. 8 also relates to Histotype. FIG. 8A shows reporting. FIG. 8B shows bit maintenance.

### DETAILED DESCRIPTION OF THE INVENTION

The invention is described in more detail below.

I. OUTSOURCE HLA-SBT/OUTSOURCE SBT

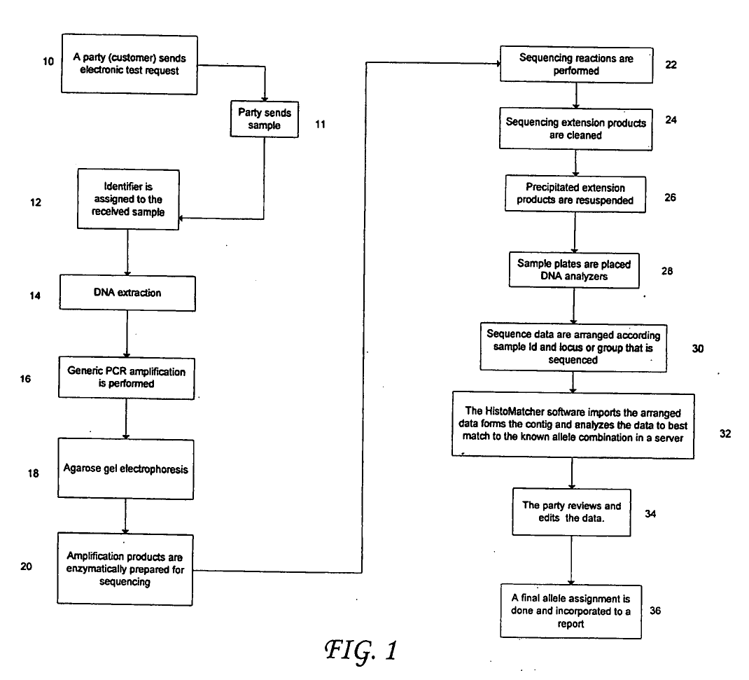

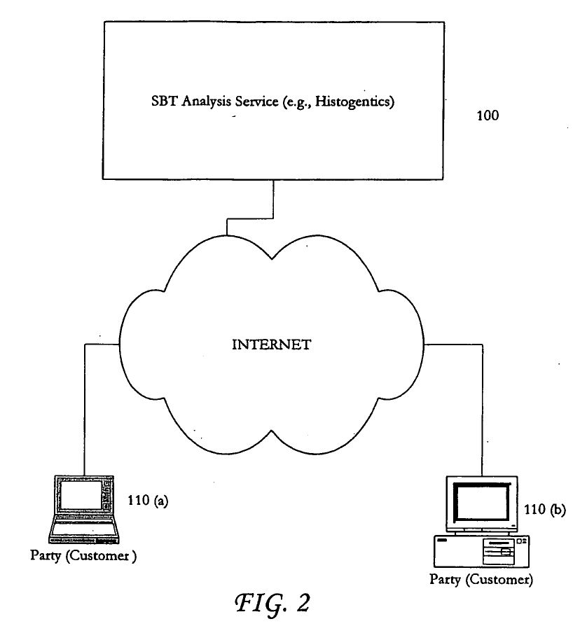

Outsource HLA-SBT/Outsource SBT is a network based system and method permitting a party to access, and interact with inventions directed toward sequence analysis and software to accomplish the same. In an exemplary embodiment, the system and method is shown via the following steps, illustrated in FIG. 1.

First, the party sends an electronic test request 10 prior to sending tissue or blood or DNA sample. Second, the party sends the sample 11 and upon receipt an identifier is assigned to the sample 12 (e.g., a tracking number) for received samples. Third, DNA extraction 14 is performed, for example by using in house proprietary protocols. Fourth, Generic PCR amplification is performed 16 for HLA-A, B, C, DRB and DQB1. Additional amplifications are performed for HLA-A, B, C, DRB1 and DQB1 subgroups. Fifth, Agarose gel electrophoresis 18 can be performed to quality control the amplifications. Sixth, the Amplification products are enzymatically prepared for sequencing 20 using Exonuclease I and Shrimp Alkaline Phosphatase cocktail. Seventh, sequencing reactions are performed 22 with ABI BigDye V3.1 chemistry, using primers extending to exons 2 and 3 for A, B, C and exon 2 for DRB1 and DQB1. Eighth, the sequencing extension products are cleaned 24 by sodium acetate/EDTA/Ethanol precipitation. Ninth, the precipitated extension products are resuspended 26 in water containing 0.01 mM EDTA. Tenth, sample plates are placed DNA analyzers 28 (e.g., ABI 3730x1 DNA analyzers). A party using the present invention can track the submitted samples from the second 10 to the tenth steps 28.

Upon completion of sequencing electrophoresis runs sequence data are arranged according sample Id and locus or group that is sequenced 30. Next, the HistoMatcher software, described below, imports the arranged data forms the contig and analyzes the data to best match to the known allele combination in a server 32. A party suitably equipped with a computer can remotely access this data via web browser (e.g., Internet Explorer, Mozilla Firefox, etc.) and review and edit the data 34. A final allele assignment is done and incorporated to a report 36. A party can also generate reports via access to the software.

II. HISTOMATCHER

HISTOMATCHER™ is a custom designed web based tool developed for Sequence Based Typing analysis. The technology behind the analysis method is to perform a point-to-point physical comparison of single and bi-directional DNA sequence traces generated by the sequencing analyzer. Based on the presence or absence of DNA variants in sample traces when compared to reference trace, the differences, also called mutations, are established. The mutations are again compared with the predetermined mutation list for different allele combinations in order to get the exact or closest matching allele combination.

The following are the highlights of this tool.

Automatic Arrangement of Sequencing Raw Data Files

Once the sequencing experiment is done and the raw data files are created, this feature will automatically organize them into a central location based on the sample id number and the experiment id. This feature requires no user interaction.

Automatic Project Creation and Mutation Detection

After the raw data files are organized, this feature will group them based on the sample id number, locus group and exon. After grouping, depending on the locus group, the reference trace and the sample files will be contiged (aligned) to determine the presence or absence of the DNA variants in the sample traces. The differences or mutations will be stored into the database for user review. While contiging, it will automatically log in the unmatched or very low quality sequencing experiment results to enable the user to redo the experiment.

User Review of Mutation with the Chromatogram

After the project or contig is created, it is available for the user to review. In this stage, the user will select an experiment, sample, group and the exon to perform a point-to-point comparison of single and bi-directional DNA sequence traces by looking at the mutation table and the chromatogram. The user will go through all the mutations detected and confirm it, edit a mutation if there is a discrepancy between both directions, delete a mutation if falsely detected and insert a mutation if not detected automatically.

Searching the Mutation Database for the Possible Allele Combination

The confirmed mutations will be compared with the custom designed table of mutations for the expected allele combination. This will display the first 500 closest match of all mutations contains the allele combination, the score and the percentage of match. The user will click a closest allele combination to check the possible mutations and review if there is a false mutation or a new mutation for that combination by clicking the mutation position.

Saving and Reporting the SBT Result

After the user reviews the closest allele combination for mutations, he/she finds the matching allele combination and saves for reporting. While saving, the system will automatically check the ambiguity and warn the user to resolve by sequencing further. After saving the final result, it will be available for reporting directly.

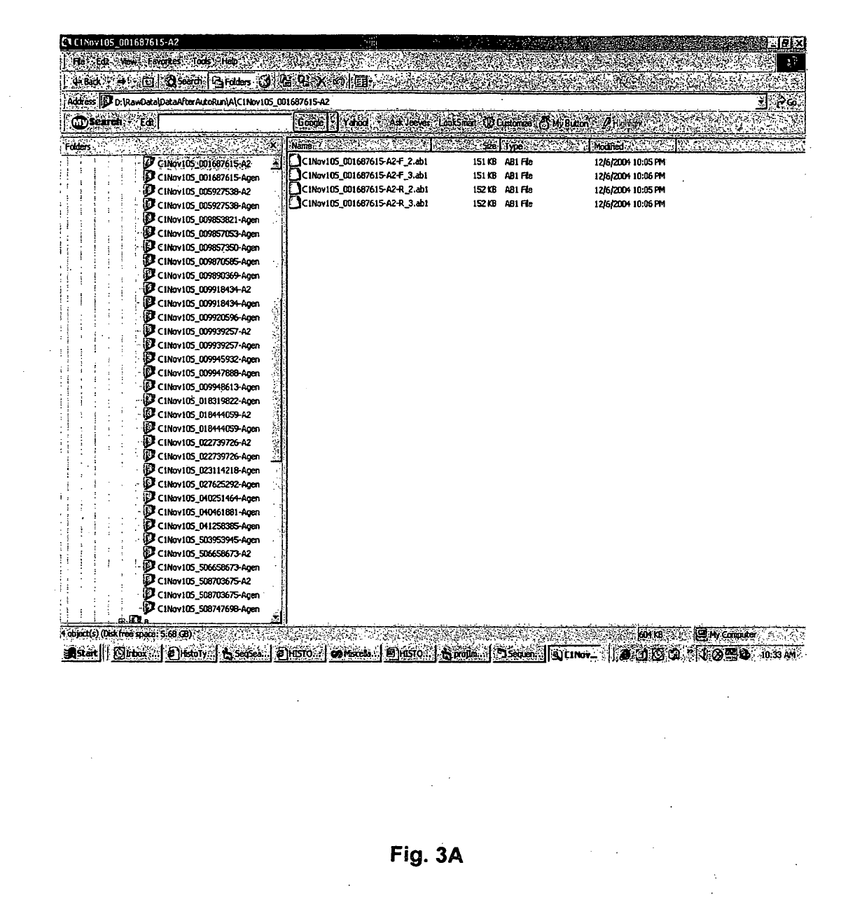

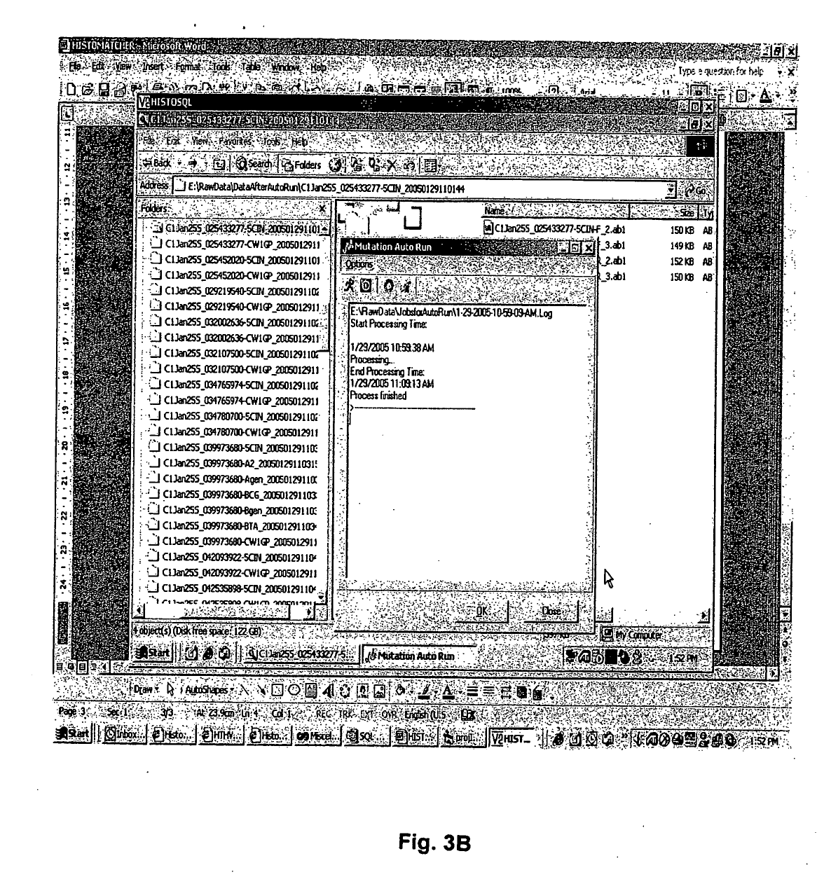

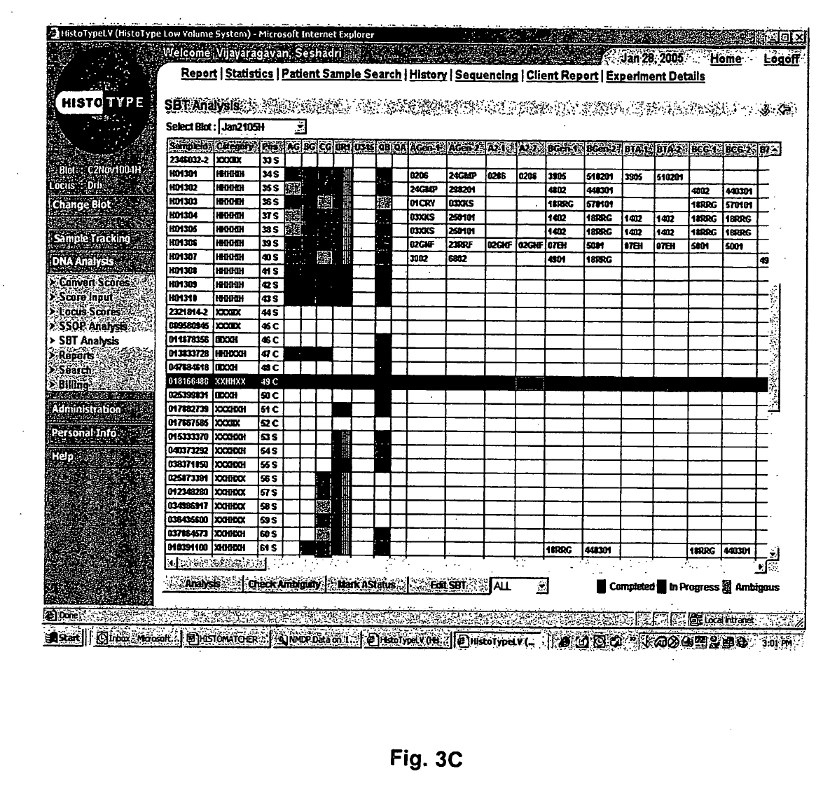

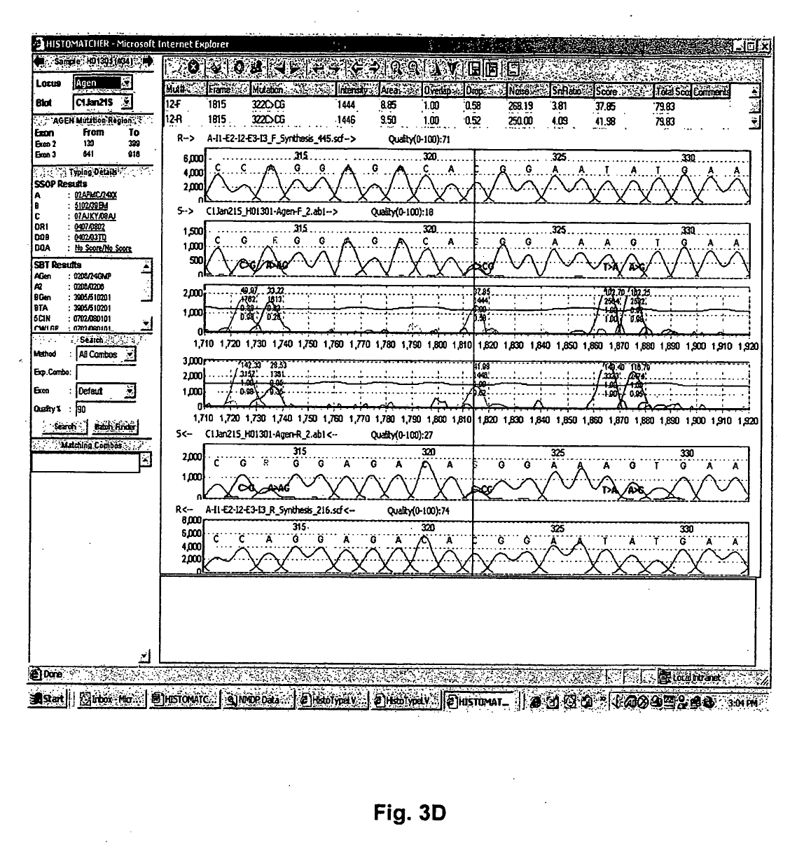

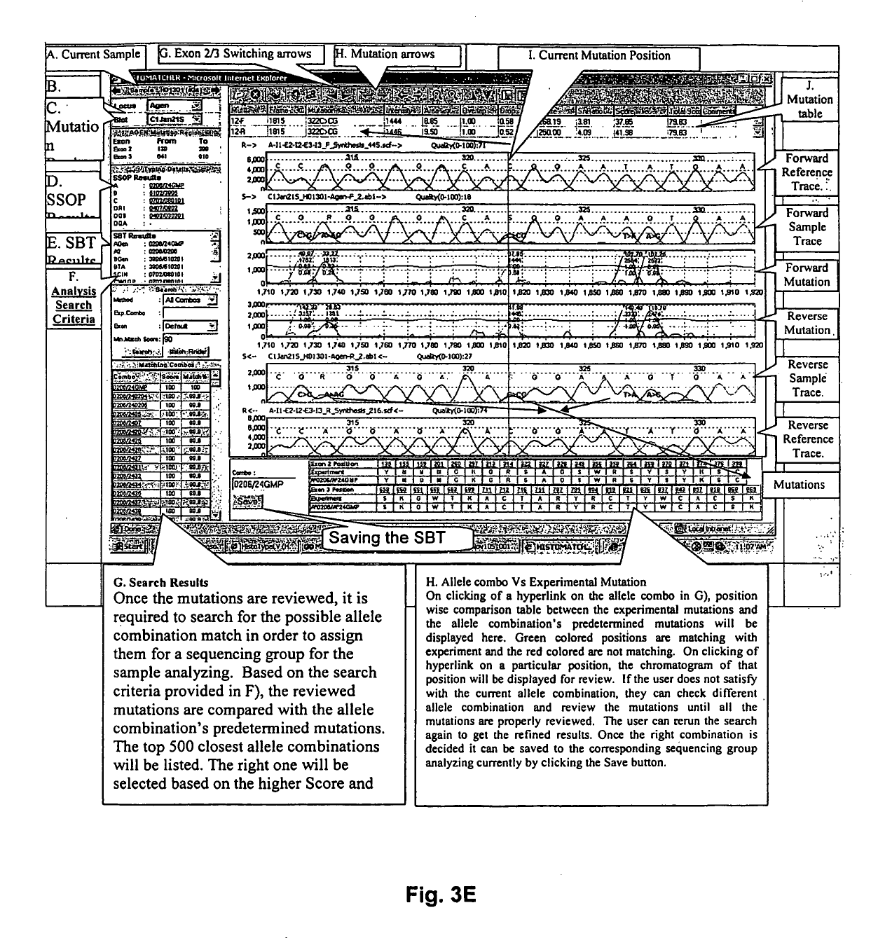

Referring to FIG. 3A, it shows the automatic arrangement of sequencing raw data files. No user interaction is required. FIG. 3B shows the automatic creation and contig calculation. Again, no user interaction is required.

FIG. 3C shows the SBT analysis main screen. It displays the Sample ID, category of typing required (A/B/C/B1, B3, B4 and B5/DQB1/DQA1), position in the plate, the status of the SBT analysis and the result of different experiments. Different color codes represent the status.

FIG. 3D shows a Histomatcher mutation review. In order to analyze or review the typing, the user will select a particular sample from screen 3) and click the Analysis button. This is the analysis screen. In this screen the user will select a particular group to analyze (by default the first sequencing group will be selected), to view the chromatogram and scroll through and review the mutations and do corrections if necessary.

FIG. 3E is a detailed view of the Histomatcher main screen. The details are as follows:
A. Current Sample: Currently analyzed sample will be displayed here. Initially this can be obtained from the Screen 3) but the user can navigate further using the arrow keys to go to the next or previous samples according to the sampleid table in screen 3.
B. SBT Group—After the contig is calculated for a sample's locus group, this will be available for analysis. The list of all experiments done for a particular sample will be displayed here. The user can select any group from the list to review.
C. Mutation Review Regions—The user will only check the mutation positions with in the ruler/reference sequence regions. This reference sequence will vary according to the loci or group analyzed.
D. SSOP results—If SSOP analysis performed for a sample, it will be displayed here. This will be very useful for cross checking the SBT results.
E. SBT Results—The SBT results of the different group and loci of the current sample will be displayed here.
F. Analysis Search Criteria—The reviewed mutations can be searched with different criteria. Like Search based on the expected allele combinations, searching only specific exons, refine search with threshold score value etc.
G. Exon 2/3 switching arrows—This is for Class I sequencing groups to review the mutations of Exon 3.
H. Mutation Arrows—On clicking of this, the chromatogram of the clicked mutation position will be displayed with the red colored line mark.
I. Current Mutation Position—Correspond to the top row on the mutation table with red color mark.
J. Mutation table—will be automatically filled by the system after running the screen 2) automatically.

In addition to the above, there is a G-Search Results. Once the mutations are reviewed, it is required to search for the possible allele combination match in order to assign them for a sequencing group for the sample analyzing. Based on the search criteria provided in F), the reviewed mutations are compared with the allele combination's predetermined mutations. The top 500 closest allele combinations will be listed. The right one will be selected based on the higher Score.

Also there is an H-Allele combo Vs. Experimental Mutation. On clicking of a hyperlink on the allele combo in G-search Results, position wise comparison table between the experimental mutations and the allele combination's predetermined mutations will be displayed here. Green colored positions are matching with experiment and the red colored are not matching. On clicking of hyperlink on a particular position, the chromatogram of that position will be displayed for review. If the user does not satisfy with the current allele combination, they can check different allele combination and review the mutations until all the mutations are properly reviewed. The user can rerun the search again to get the refined results. Once the right combination is decided it can be saved to the corresponding sequencing group analyzing currently by clicking the Save button.

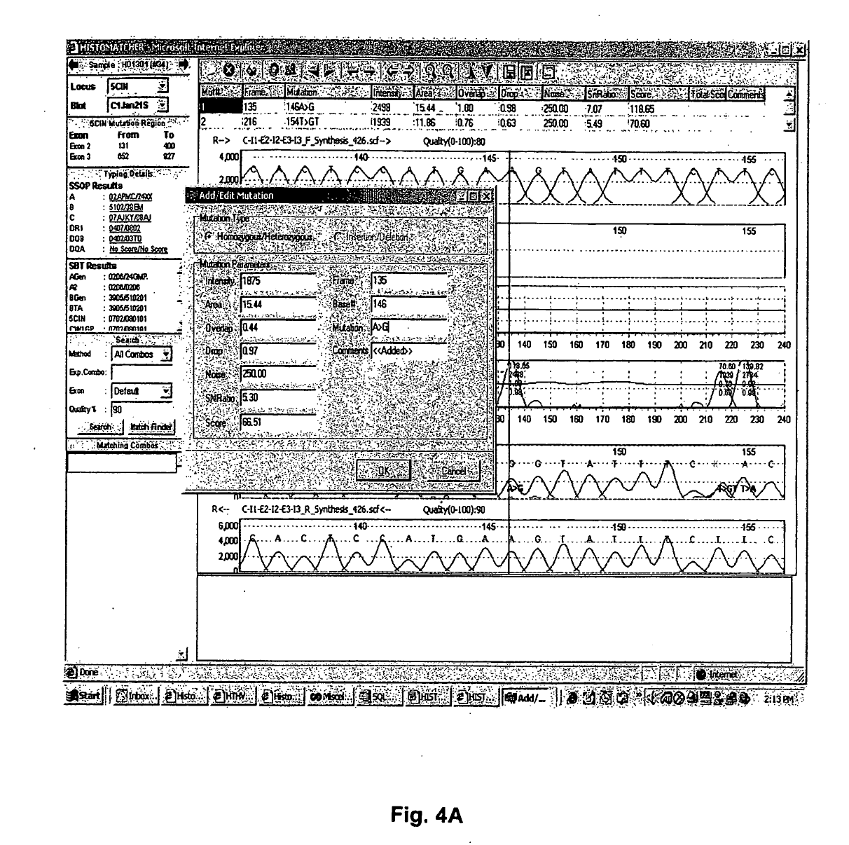

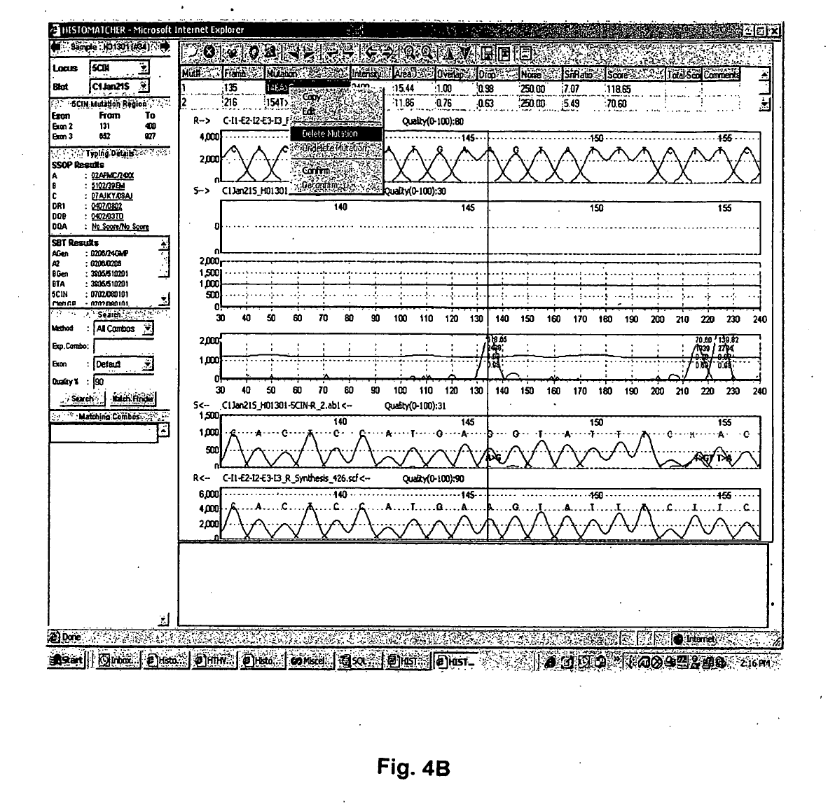

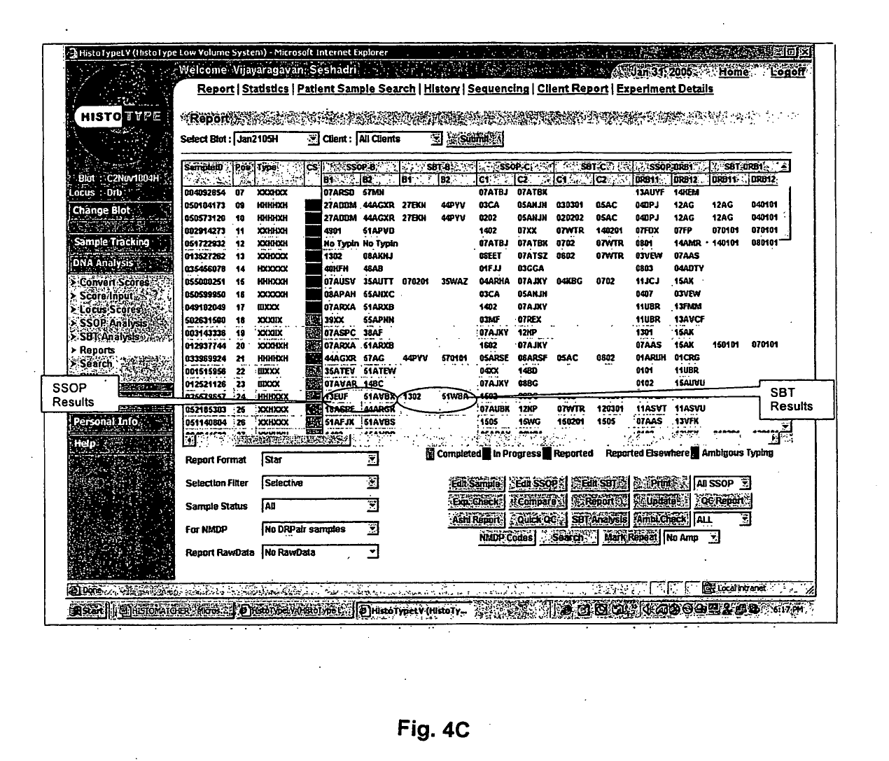

Referring now to FIG. 4, FIG. 4A shows how to edit a mutation. To edit a mutation, place the cursor anywhere on the mutation peak in the electropherogram. Click control key together with left mouse key. A popup window appears as above along with the position and the mutation. Simply and Edit and Click OK to save.

FIG. 4B shows how to confirm a mutation. To confirm a mutation, go to the position in the mutation table, right click the mouse and select “Confirm Mutation” option.

FIG. 4C shows the reporting. Once the SBT data is saved, it is available for reporting directly. Depending on the resolution of the request, it can be reported. Different color codes represent the status of the reporting.

III. HISTOTIE™

Introduction:

HistoTie is a web-based application, which ties Sequencing and SSO results. Using the data obtained from SSO, the results of sequencing can be quality controlled and similarly using the data obtained from sequencing SSO results can be verified. It also helps as a tool for Quality assurance.

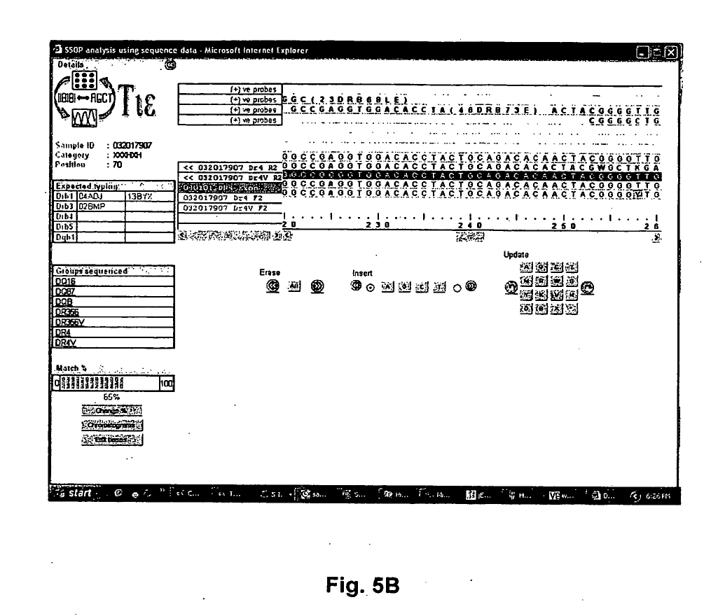

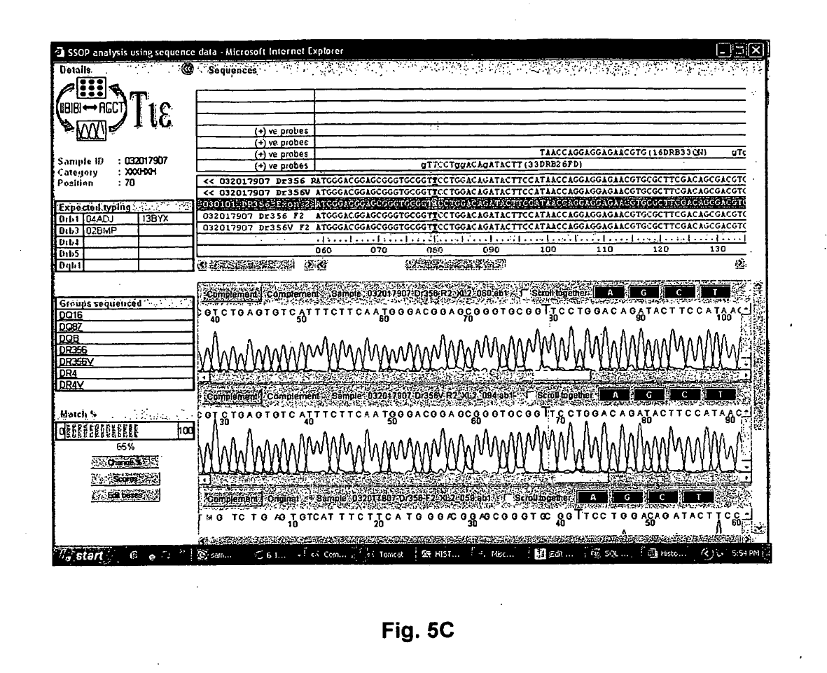

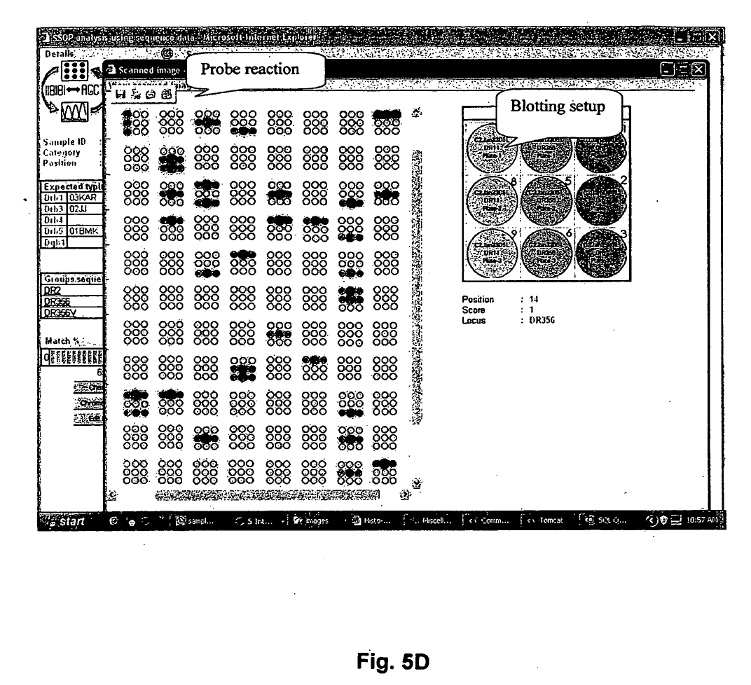

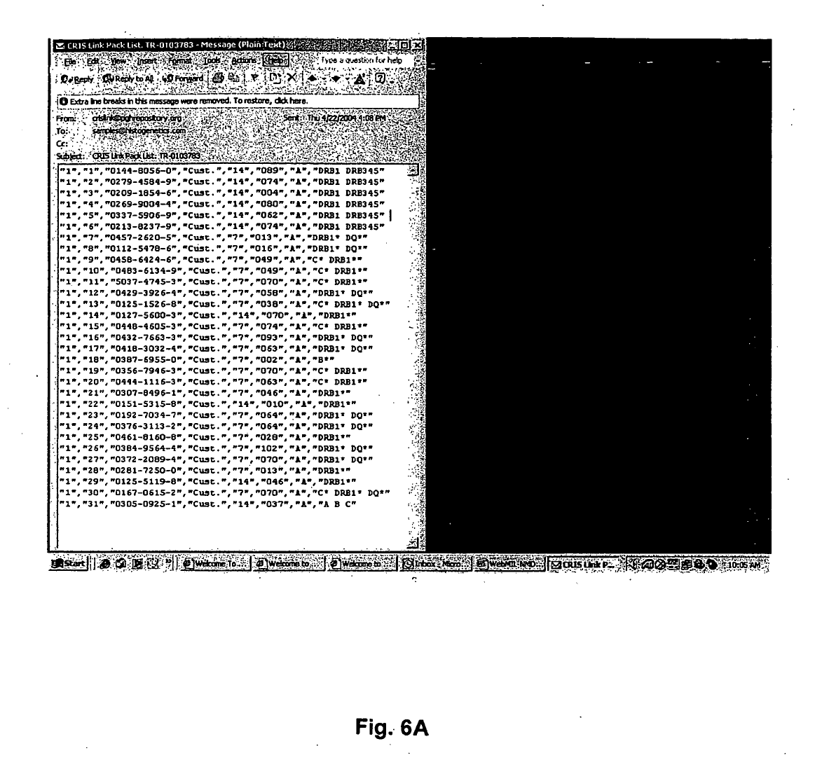

FIG. 5A shows the main window of HistoTie™.

Reproducing SSO Result from Sequencing Data

Forming Contigs:

Based on the groups sequenced, contigs are formed on the fly when a sample is selected.

The program displays the reverse and forward sequences aligned to a ruler with the list of positive probes aligned on the top of the sequencing data. Based on the data obtained from the ABI file, the list of positive probes is determined. The program checks either the forward or the reverse sequence or both to determine if a probe can be positive.

The score thus obtained and the score obtained by SSO is compared and displayed. The user can click on the scores that do not match, to see the region where the mismatch occurs. The program uses the list of probes from the current kit to determine the score.

Editing the Bases:

The bases can be edited and corrected, if the base calling is incorrect. FIG. 5B shows the edit mode screen. Bases can be inserted, deleted and updated. The application is by default in view mode, Click on the ‘Edit’ button to go to edit mode and highlight the base(s) that need to be corrected and click on the appropriate button to make the change.

Viewing the Chromatogram:

The chromatograms of the samples can be viewed to correct the incorrect base calling. To view the chromatogram, click the ‘Chromatogram’ button. One example is shown in FIG. 5C.

Viewing Probes that are not Positive:

Only the positive probes are aligned and displayed on top of the sequences. The probes that are not positive (negative probes) are displayed in a separate list. When the user selects a negative probe, the sequences at that probe region and the probe sequence are displayed as a proof that the probe cannot be positive. A probe reaction view is shown in FIG. 5D, which shows a probe reaction on the left and a blotting setup on the right.

IV. HISTOTYPE

HistoType™ is a proprietary software developed for Sample Tracking, Data handling, SSOP Typing Analysis and Database Management. It's a web based digital nervous system solution that helps the lab to provide superior customer service by delivering very precise and accurate report on time. It keeps track of the samples and stand behind the samples from the moment they arrived at the lab till it is being reported. The following are the hierarchical process:

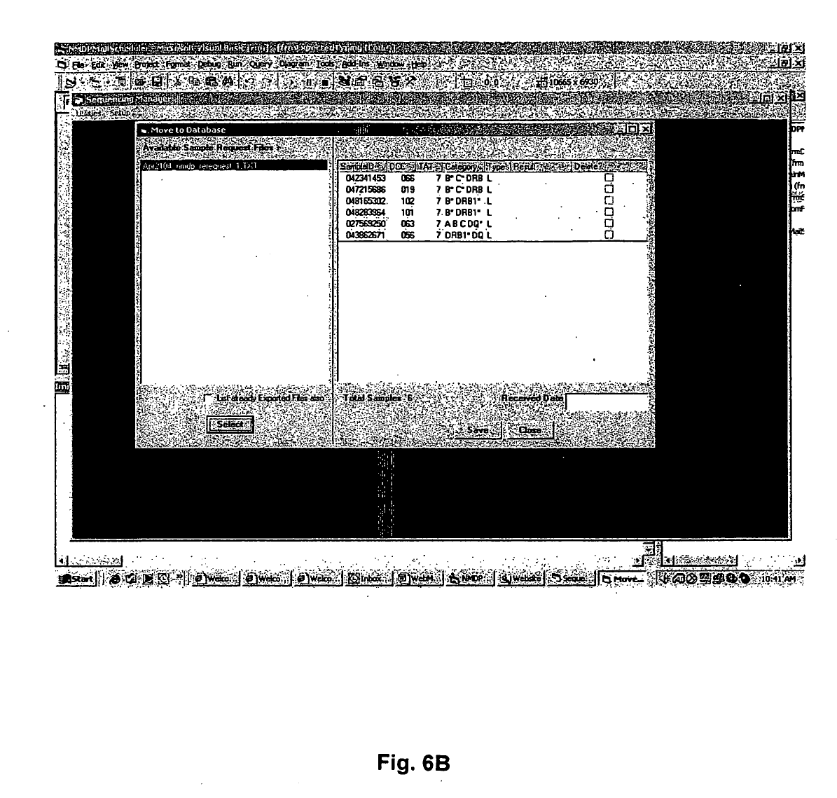

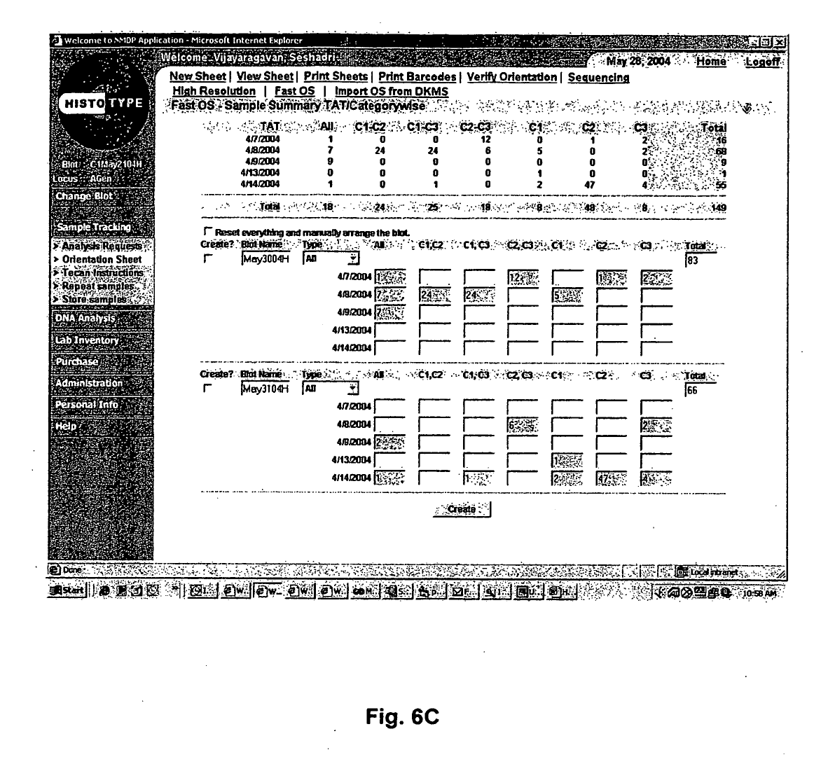

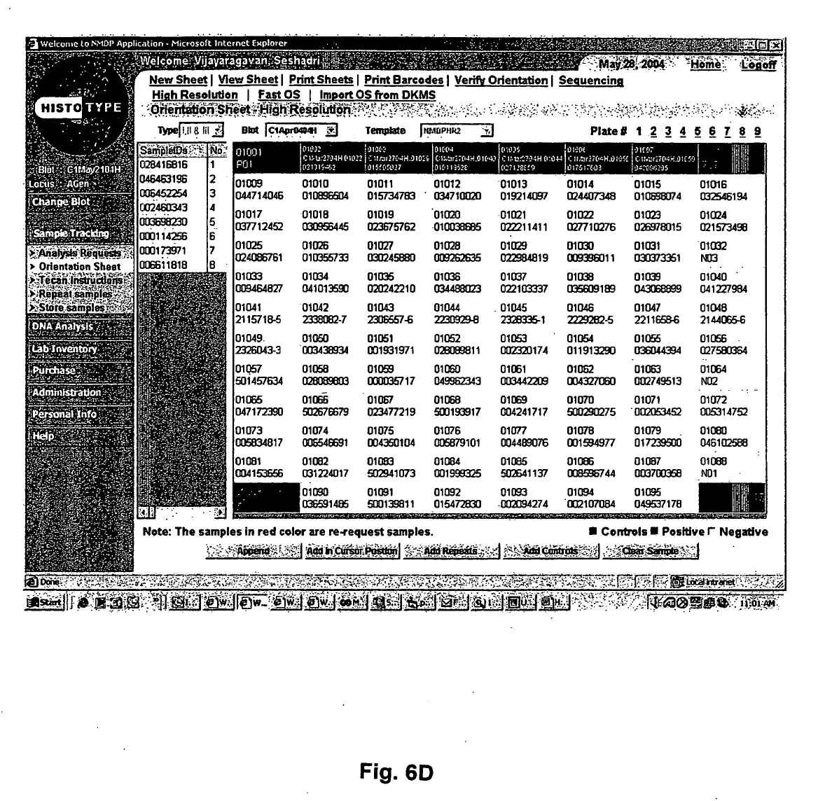

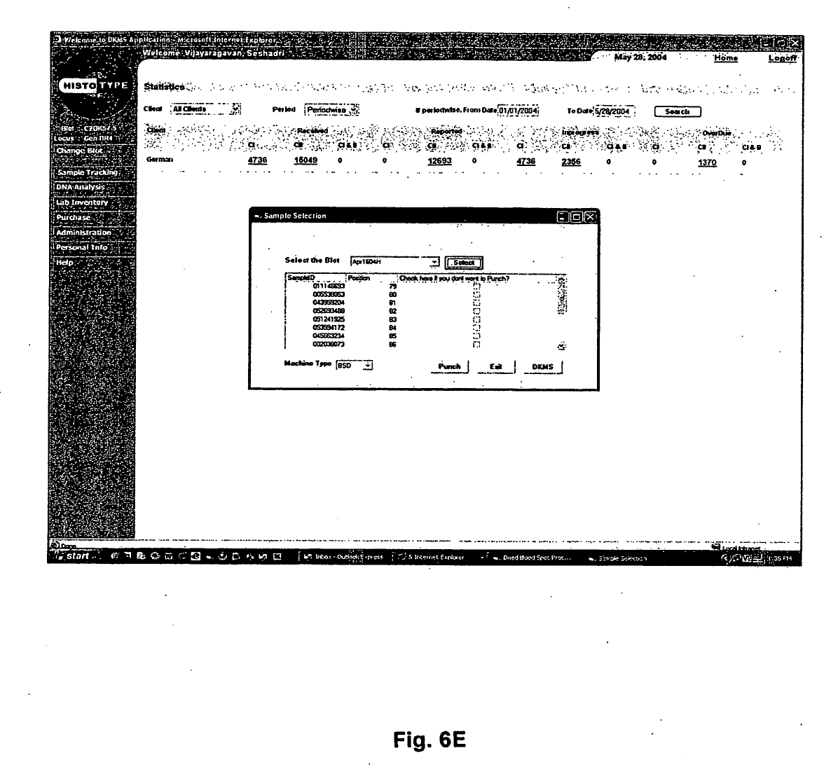

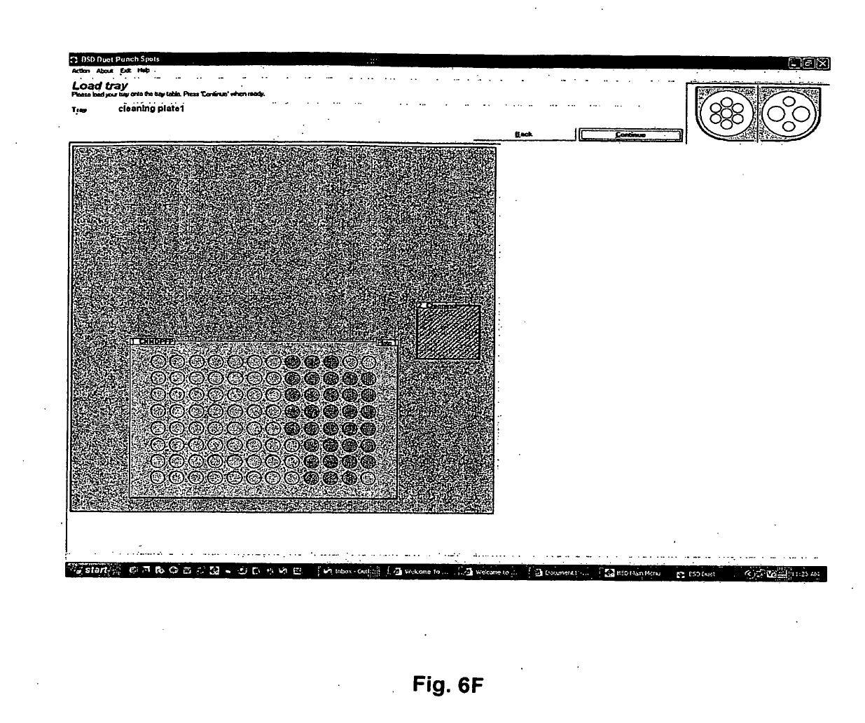

1. Typing Request
NMDP will send their typing request by email in a fixed format. This is shown in FIG. 6A. This email contains the sample information like Sample ID, donor center code, typing category etc.
As shown in FIG. 6B, the MailScheduler program will import the sample information from the email into the HISTOTYPE system. Once the sample is imported into the system, it will be ready for experiment.

2. Orientation Sheet
Grouping and arranging the samples received from NMDP into a 96-well micro titer plate. This is shown in FIG. 6C.

FIG. 6D shows a sample 12×8 orientation sheet. Included are:
Adding the Controls for quality control.
Generating the script for Tecan to transfer the blood samples from vials.
Generating the script for Dried Blood Processor for filter paper sample punching
Verify the orientation after manual arrangement in the plate
Confirming the Orientation Sheet
Automatic Probe Kit assignment to a given locus for each amplification.

FIG. 6E shows filter paper punching script generation, and includes generating the script for cherry picking of the ambiguous typing samples for sequencing to resolve the ambiguity.

FIG. 6F shows filter paper punching using the DBS. The script will ensure that the correct sample is punching in the correct position.

3. Score Input and Analysis

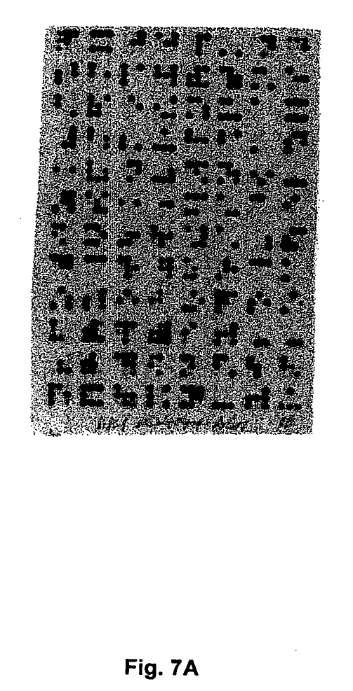

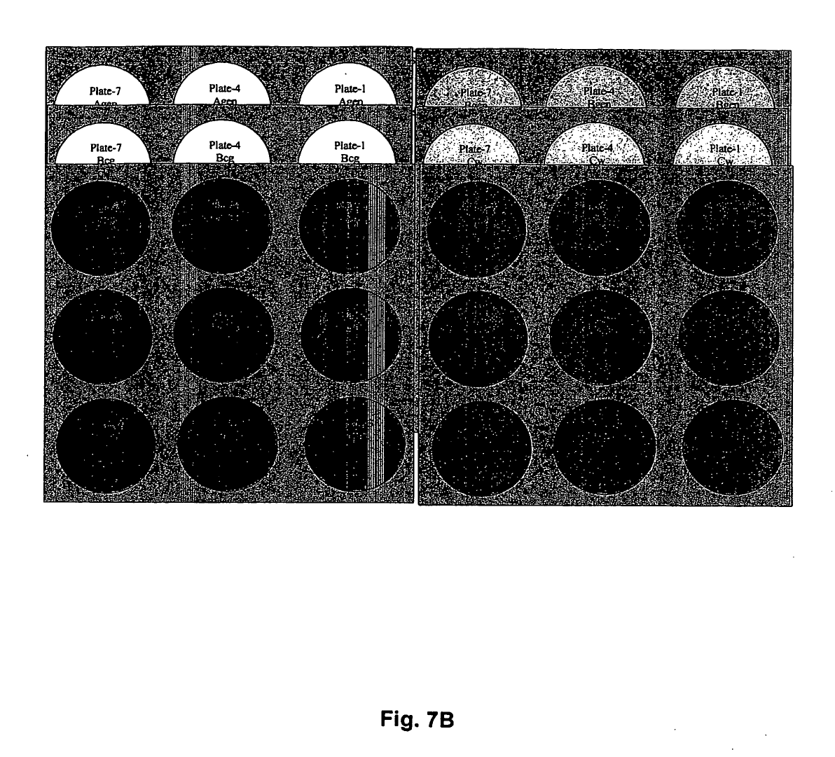

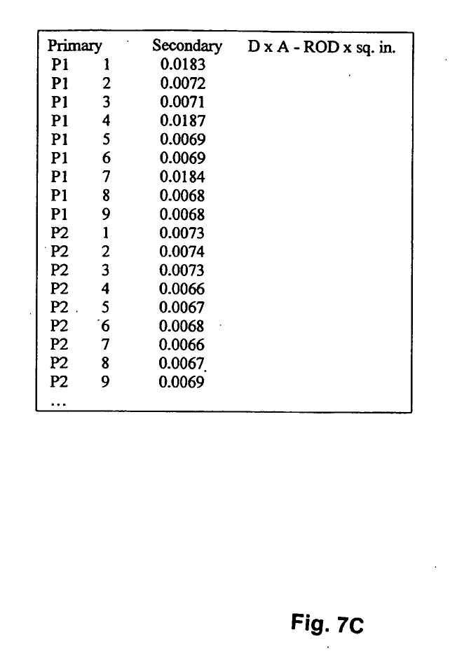

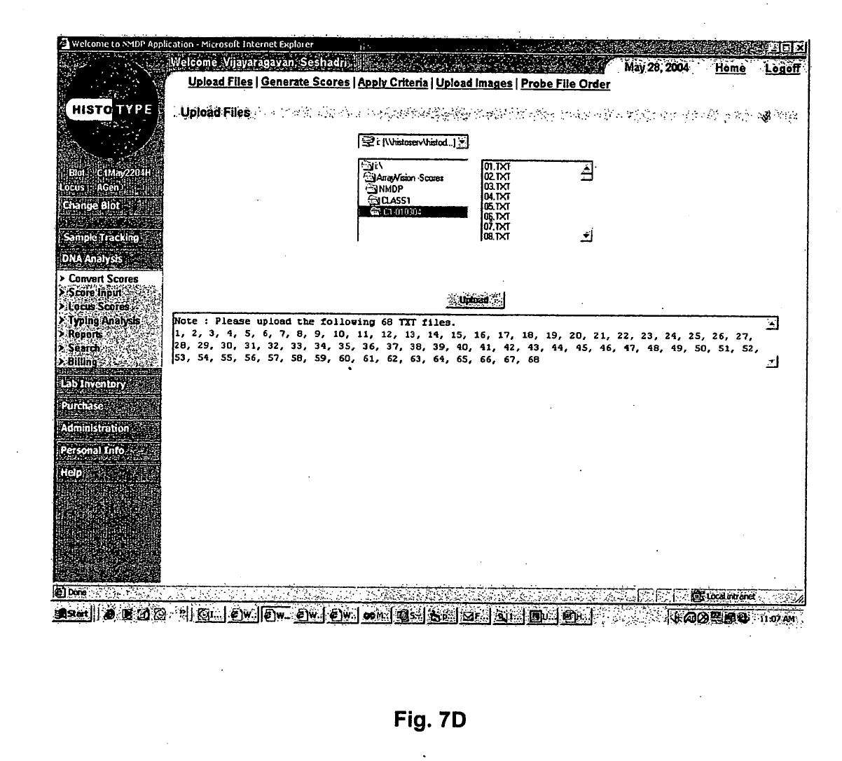

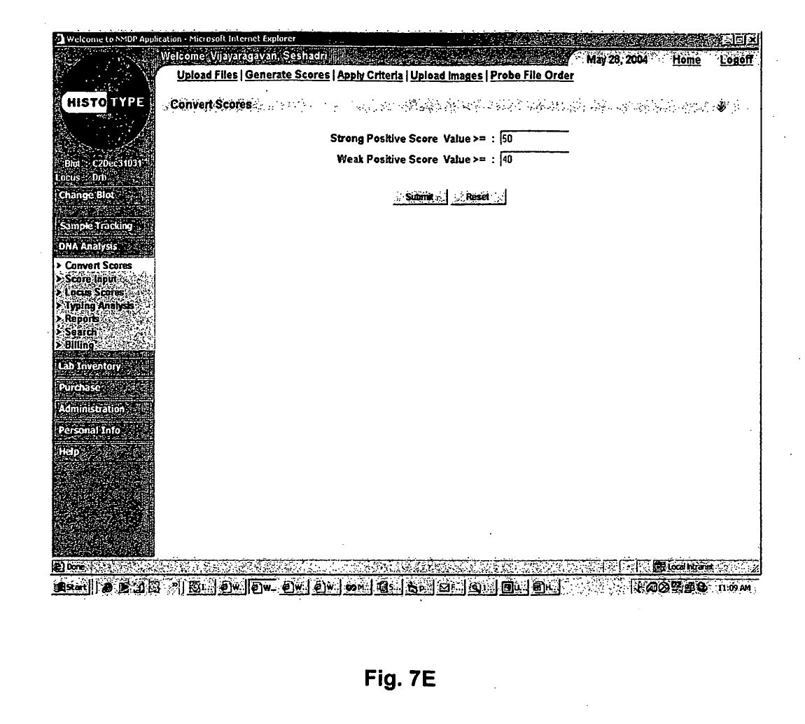

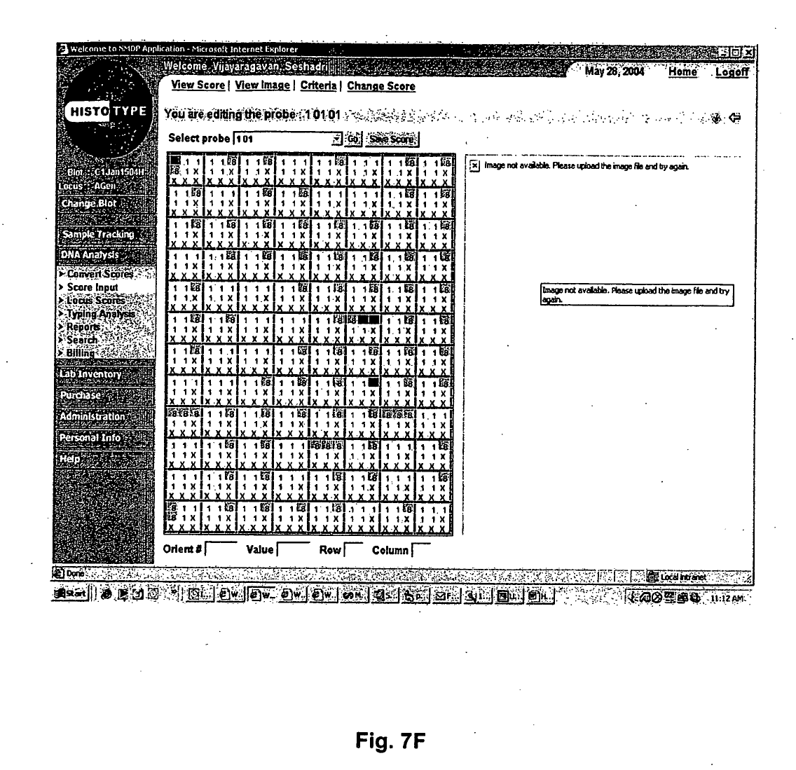

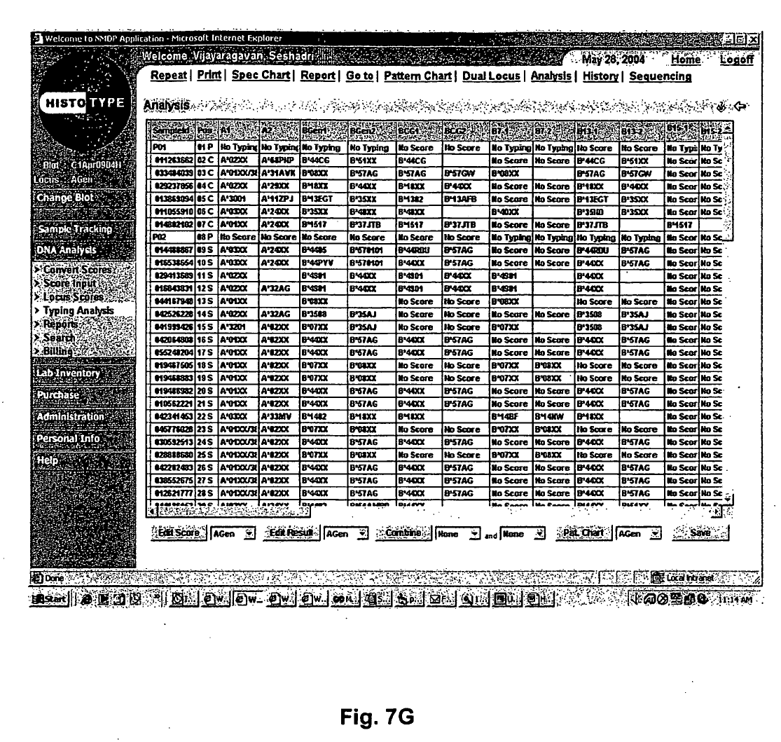

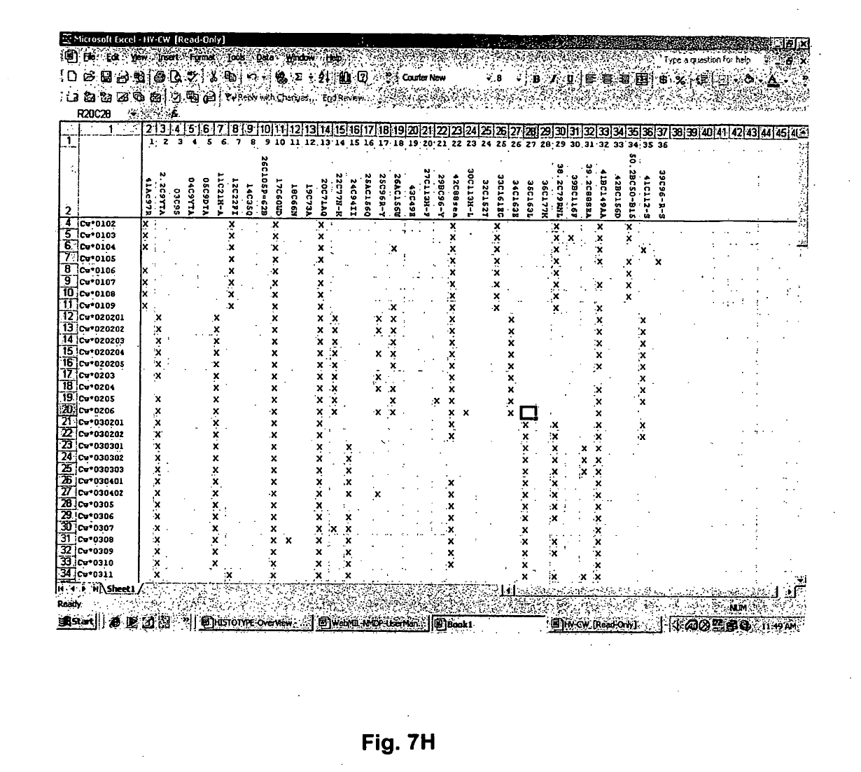

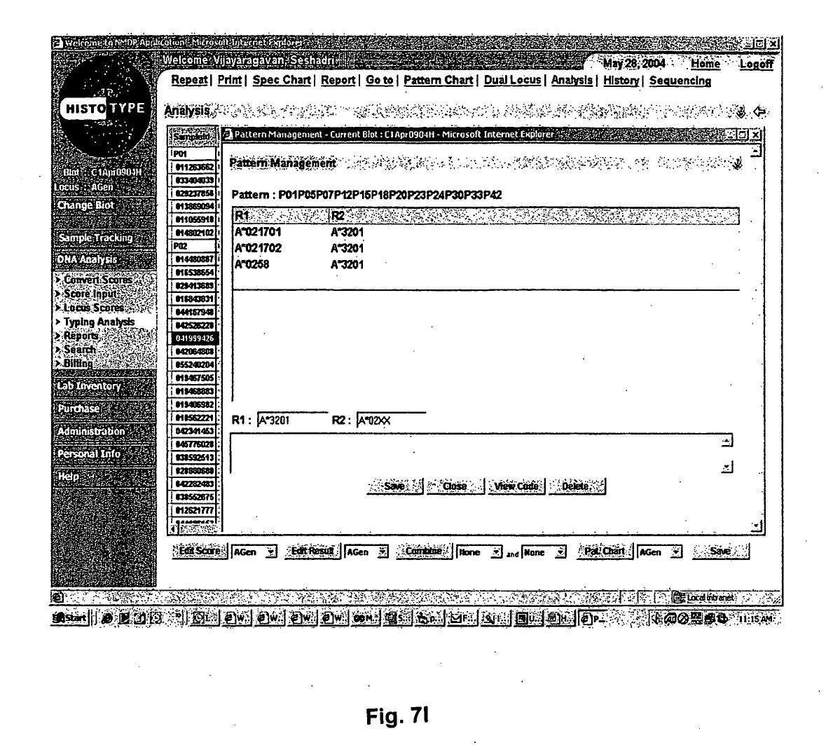

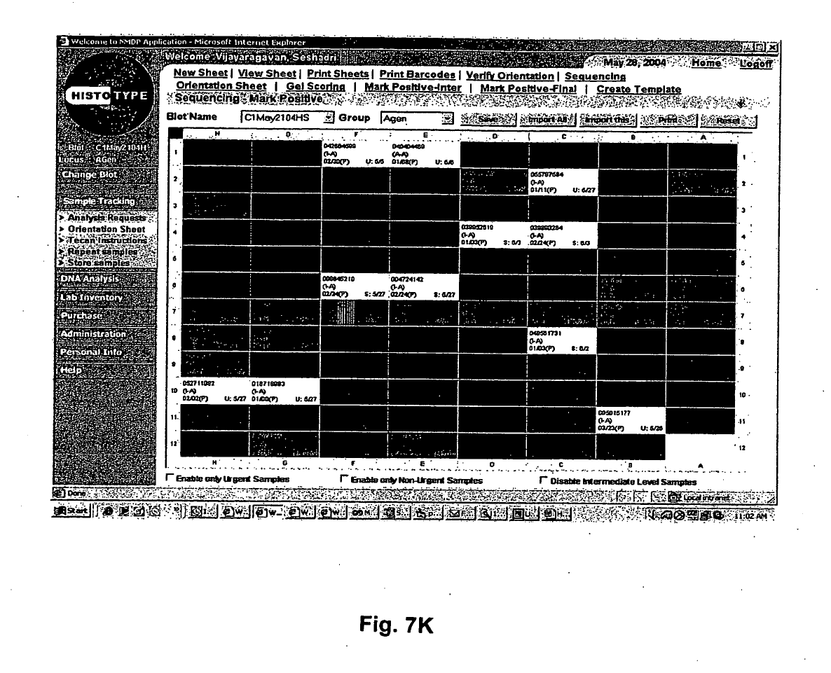

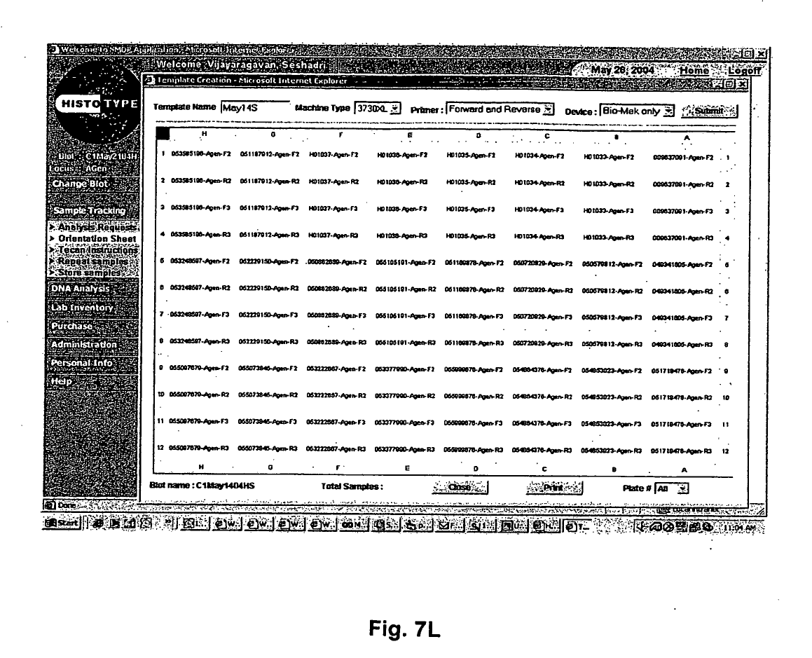

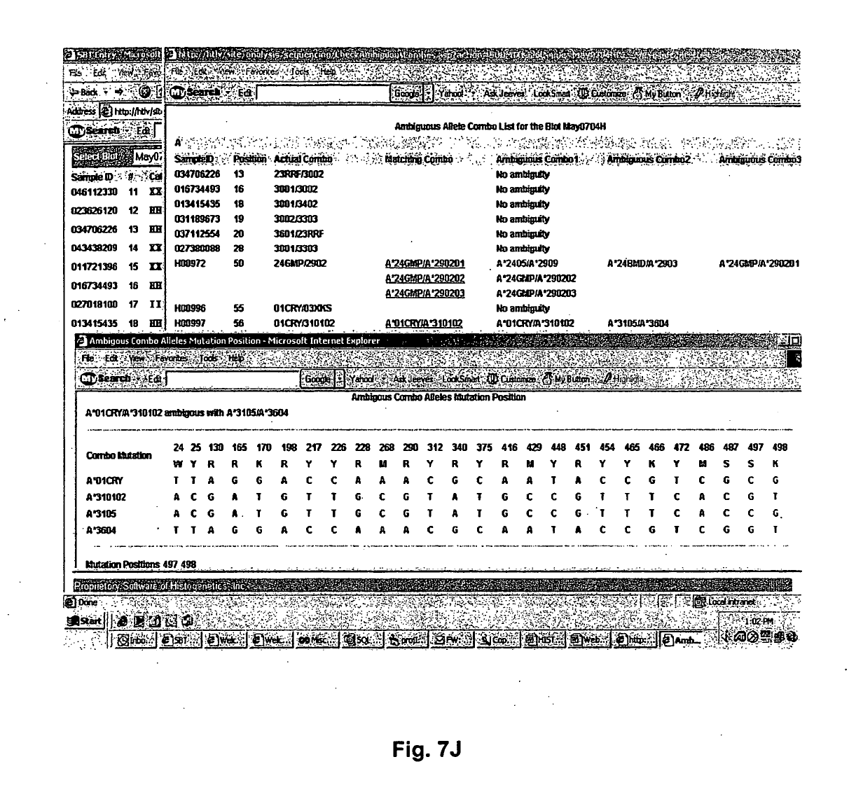

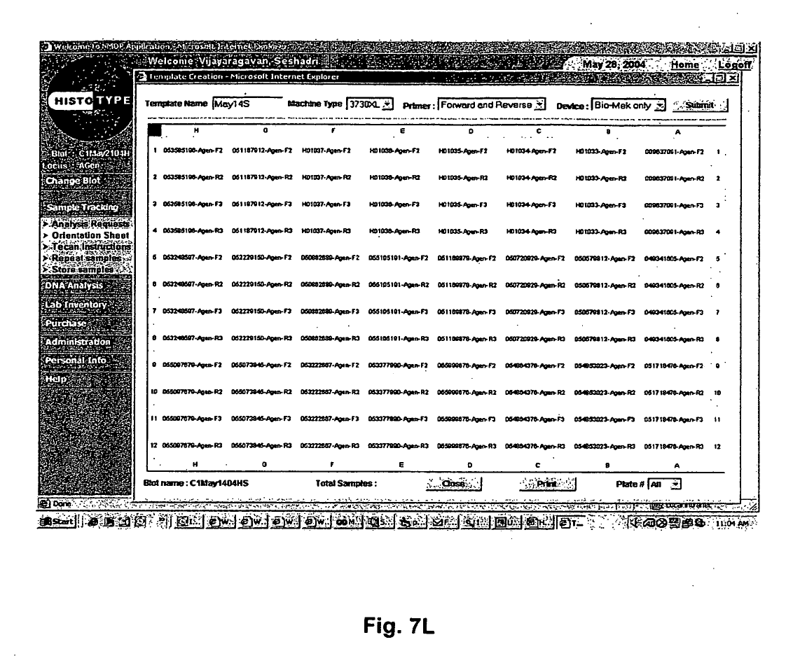

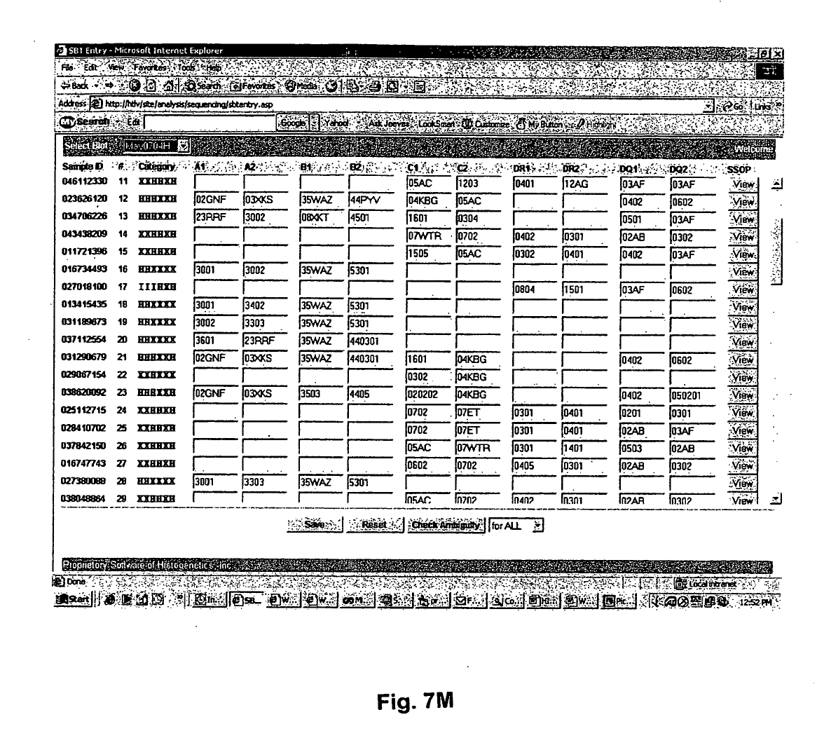

This is shown in FIGS. 7A through 7N and involves:
Importing the probe reaction data scores created using the array vision software for all the probes in the locus kit.
Identifying the probe hit (Positive and Negative reactions) for all the samples and for all the probes in the kit by applying the threshold score range.
Analyzing each sample to determine the allele combinations and generating automatic allele codes typed by the probe kits for all the loci requested. Resolving ambiguity by analyzing with sub-groups.
Reviewing the allele assignment from Pattern Chart.
Combing Sequencing data to SSOP data and vice versa.
Ambiguous combination checking.

Referring in more detail to FIGS. 7A and 7B, after hybridization process, the developed probe reaction film will look like FIG. 7A. This is for a probe. Similarly one will have probe reaction films available for all the probes of a locus kit. It is a probe reaction film for 864 samples arranged in 9×12×8 micro plate. It has 12 rows and 8 columns. The position starts from top right through left and goes down. For example the Position 1 starts on top right and 8 is on top left and position 89 is on the bottom right and 96 is in bottom left corner. Each dot represents a sample. The intersection of a column and a row in the above picture has 3×3 form to accommodate all the 9 plates in an experiment blot. For example the top right corner has 1^{st} position of all the 9 plates arranged in 3×3. See FIG. 7B for the blotting arrangement. In this method of blotting, totally 864 samples can be processed in a test.

Probe reaction scores are shown in FIG. 7C. Each probe reaction films will be scanned and saved as a TIFF file to identify the dark and light intensity spots using the Arrayvision software. The Arrayvision will generate the probe reaction score as a tab delimited text file for each tiff file. It will look like above.

FIG. 7D shows importing probes reaction score into the HistoType. It is required to import the text files generated using the arrayvision into the corresponding Blot and locus of the HistoType system in order to convert the intensity fractional values into a normalized positive/weak positive and negative values (8/4/1) using the custom algorithm. The number of text files (probes) for the locus to be imported will be defined in the blot locus kit. The blot locus kit will be assigned automatically during the confirmation of a blot.

Next, FIG. 7E shows applying threshold % range to find +ve, −ve reactions. In this step, all the probes reaction scores will be converted as 8/4/1 (positive/weak positive/negative) based on the threshold range given. If a fractional value is above this range, then it is positive, if it is between this range, then weak positive and if it is below the range then it will be considered a negative.

FIG. 7F shows a review of the converted probe reaction scores. Positive reaction samples will be in green color, the weak positive in orange and the negative in black color. This is exactly corresponds to probe reaction film. The user has to review each probe and change if necessary. They can re-apply the threshold range (criteria) for a probe, or manually edit the score if necessary.

FIG. 7G shows an analysis, and FIG. 7H shows a sample probe bit pattern in Excel. After the probe reaction score review, the probe hit scores will be available for all the sample of a locus in an experiment blot. For example, for ‘AGen’ Locus, a sample's probe score will look like 881181118881111118811811811188881.

It has totally 38 probes reaction starting from left through right. 8 represent the positive reaction and 1 represents the negative reaction. This score will be converted as probe hit patterns and compared with the allele probe hit database to get the allele combination. It generates and assign the NMDP allele code in case of more than one allele combination hits the required pattern.

For example the probe hit pattern for the above score is P01P02P05P09P10P11P18P19P22P25P29P30P31P32.

The allele assignment for the above pattern will be A*01XX/A*11AA. This can be obtained from the standard algorithm (The allele combination's combined probe hit is the same as our required pattern). Similarly the analysis will be done for all the samples in a test. The user can do either Whole batch Analysis or Selective Analysis. Once the typing is available for all the locus requested for a sample, it will be ready for reporting after ambiguous checking.

FIG. 7I is a pattern chart of a sample's locus. Here there are two sections. On the top table is the different allele combination which satisfies the required pattern and the bottom is the NMDP code assignment for the pattern.

After the analysis using SSOP method, the ambiguous allele combination samples will be identified and further analyzed using sequencing based methods. This is shown in FIG. 7J.

FIG. 7K shows generating the script for cherry picking of the ambiguous typing samples for sequencing to resolve the ambiguity. FIG. 7L shows adding sequencing primers and creating .PLT file for each sequencing plates for the 3730 Analyzer.

After analysis by SBT method, the result will be entered using the step as shown in FIG. 7M. It is required to combine the SBT data with SSOP for reporting. This can be done as shown in FIG. 7N.

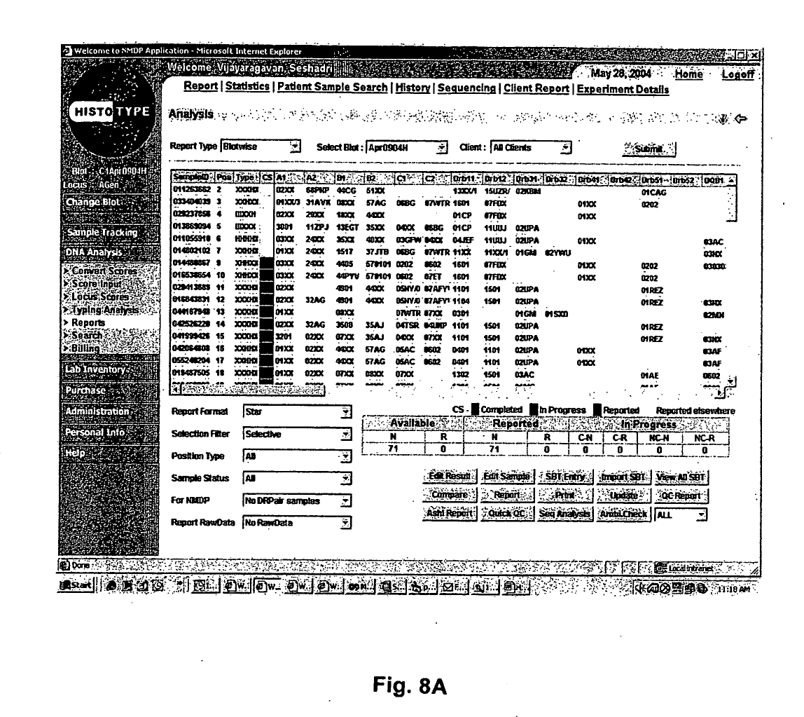

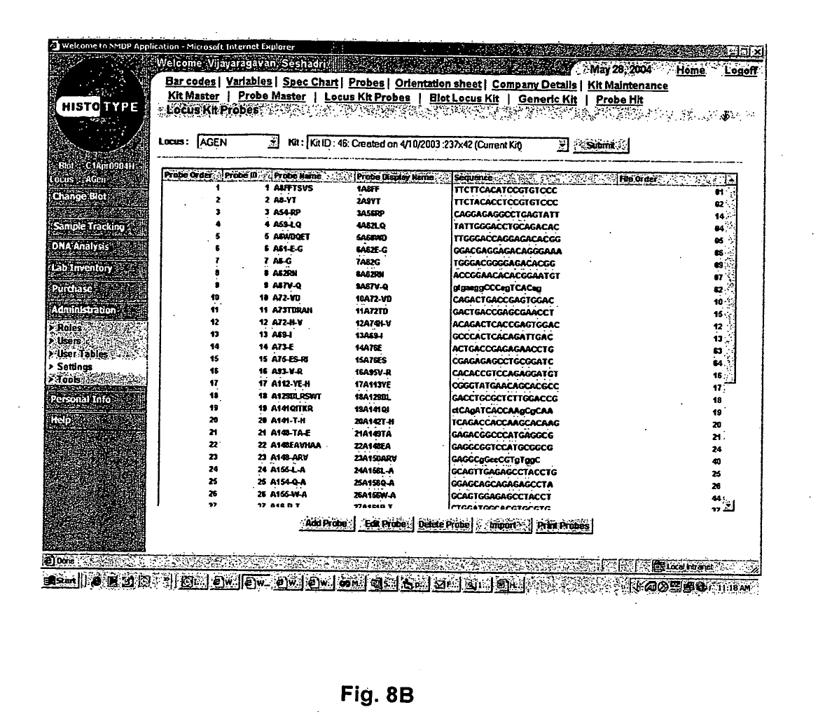

4. Reporting
Analyzed and Completed samples reported as per the client's requirement. This is shown in FIG. 8A.

5. Administration
Set up and Maintain the Master Probe List (Probe Master)
Creating and Managing probe kits (Kit Master)
Setting up the current kit for all the loci (Locus Kit Probes)
Re-assign the kit to the blot and locus if required (Blot Locus Kit)
Creating the Allele Probe Hit (Probe Hit)
Updating the NMDP Allele Code (Probe Hit)
Setup and maintain the roles, users and their rights in the program
FIG. 8B shows kit maintenance.

### Overall Picture of the HISTOTYPE System with Screenshot

HistoType is our proprietary software developed for Sample Tracking, Data handling, SSOP Typing Analysis and Database Management. It is a web based digital nervous system solution that helps the lab to provide superior customer service by delivering very precise and accurate report on time. It keeps track of the samples and stand behind the samples from the moment they arrived at the lab till it is being reported. The following are the hierarchical process:

Typing Request Arrival from Email and Importing into HistoType system.
↓
Grouping and arranging the sample in 9 × 12 × 8 micro plate format
called Orientation Sheet in the HistoType system and assign the name
to the experiment called BLOT.
↓
Manually arrange the sample according to the orientation sheet and
perform the experiment.
↓
Verify the manual arrangement through HistoType, confirm the blot and
locus kit assignment.
↓
Import the Probe Reaction Scores after Hybridization (Score Input)
↓
Probes reaction score review
↓
Perform the Analysis
↓
Detect the ambiguous combination samples and resolve by SBT method
↓
Combining SBT and SSOP data
↓
Report

It will be readily appreciated by those skilled in the art that modifications may be made to the invention without departing from the concepts disclosed in the foregoing description. Accordingly, the particular embodiments described in detail herein are illustrative only and are not limiting to the scope of the invention, which is to be given the full breadth of the appended claims and any and all equivalents thereof.

1. A method of accessing analyzed a nucleic acid sample which comprises:
a) receiving an electronic request to process a nucleic acid sample;
b) receiving said nucleic acid sample;
c) applying an identifier to said nucleic acid sample;
d) inputting data concerning said nucleic acid sample into a computer;
e) manipulating said data with software;
f) generating results from said manipulation;
g) wherein a party can access said data and said results through a network via a remote computer.

2. A method for accessing and manipulating sequence data generated in a sequence analysis of a sample, the method comprising the steps of:
receiving a request for the sequence analysis of the sample from a party;
assigning an identifier to the sample;
arranging the sequence data according to the sample identifier;
forming a contig in accordance with the arranged data;
analyzing the data to match the data to a known allele combination;
performing an allele assignment; and
reporting a sequence analysis result, wherein
tracking data regarding the sequence analysis is available to the party, and
the sequence data is available to the party for reviewing and editing by the party.

3. A method according to claim 2, wherein said analysis is performed by a sequence based typing (SBT) analysis service, and the party communicates with the analysis service using a network based system.

4. A method according to claim 3, wherein said step of receiving a request comprises receiving an electronic request from the party via the network based system.

5. A method according to claim 2, wherein the result includes the allele assignment.

6. A method according to claim 2, wherein the sequence analysis comprises at least one of
DNA extraction;
PCR amplification;
agarose gel electrophoresis;
enzymatic preparation of amplification products;
sequencing reactions;
cleaning of sequencing reaction products;
resuspending precipitated reaction products; and
placing sample plates in a DNA analyzer.

7. A method according to claim 2, wherein said steps of arranging the sequence data, forming a contig, analyzing the data, and performing the allele assignment are performed by a software tool, the software tool being accessible to the party.

8. A method according to claim 7, wherein the software tool is accessible to the party for generating a report based on at least one of the sequence data and the allele assignment.

9. A sequence analysis system for performing a sequence analysis process on a sample received from a party and for generating sequence data, the system comprising:
a computer configured to execute a method including the steps of
processing a request received from a party for sequence analysis of the sample;
assigning an identifier to the sample;
generating tracking data regarding the sequence analysis process;
arranging the sequence data according to the sample identifier;
forming a contig in accordance with the arranged data;
analyzing the data to match the data to a known allele combination;
performing an allele assignment; and
reporting a sequence analysis result, wherein
the tracking data is available to the party during the sequence analysis process, and
the sequence data is available to the party for reviewing and editing by the party.

10. A system according to claim 9, wherein the computer is connected to a network based system for communication with the party.

11. A system according to claim 9, wherein the computer is configured to receive the request for analysis from the party via the network based system.

12. A system according to claim 9, wherein the sequence analysis process includes analysis by a DNA sequencing analyzer and a point-to-point physical comparison of single and bi-directional DNA sequence traces generated by the sequencing analyzer, and wherein the computer is configured to perform a comparison of the DNA sequence traces with a reference trace to determine the presence of mutations.

13. A system according to claim 9, wherein the computer is configured to arrange the sequence data automatically.

14. A system according to claim 9, wherein the computer is configured to organize the data in a central location based on the sample identifier and an experiment identifier.

15. A system according to claim 14, wherein the computer is configured to group the organized data based on at least one of the sample identifier, a locus group and an exon.

16. A system according to claim 16, further comprising a database for storing mutations.

17. A system according to claim 17, wherein the database is available for review by a user.

18. A system according to claim 16, wherein entries in the database may be edited and/or deleted by a user.

19. A system according to claim 9, wherein the computer is configured to create a project automatically in accordance with the arranged data.

20. A system according to claim 19, wherein the project is available for review by a user.

21. A computer program product for sequence based typing analysis, the computer program product comprising executable code for performing a method including the following steps:
processing a request received from a party for sequence analysis of the sample;
assigning an identifier to the sample;
generating tracking data regarding the sequence analysis process;
arranging the sequence data according to the sample identifier;
forming a contig in accordance with the arranged data;
analyzing the data to match the data to a known allele combination;
performing an allele assignment; and
reporting a sequence analysis result, wherein
the tracking data is available to the party during the sequence analysis process, and
the sequence data is available to the party for reviewing and editing by the party.

22. A computer program product according to claim 21, wherein the sequence analysis process includes analysis by a DNA sequencing analyzer and a point-to-point physical comparison of single and bi-directional DNA sequence traces generated by the sequencing analyzer, and wherein the method further includes comparing the DNA sequence traces with a reference trace to determine the presence of mutations.

23. A computer program product according to claim 21, wherein the method further includes arranging the sequence data automatically.

24. A computer program product according to claim 21, wherein the method further includes organizing the data in a central location based on the sample identifier and an experiment identifier.

25. A computer program product according to claim 24, wherein the method further includes grouping the organized data based on at least one of the sample identifier, a locus group and an exon.

26. A computer program product according to claim 21, wherein the method further includes creating a project automatically in accordance with the arranged data.

27. A computer program product according to claim 26, wherein the method further includes permitting a user to view a mutation table and a chromatogram associated with the project.

28. A computer program product according to claim 27, wherein the method further includes comparing mutations confirmed by the user with a table of mutations to determine a possible allele combination corresponding to the confirmed mutations.

29. A computer program product according to claim 28, wherein the method further includes permitting a user to review a closest matching allele combination.

30. A computer program product according to claim 21, wherein the computer program product is a web-based application.

31. A computer program product for relating SSO results to sequencing data, the computer program product comprising executable code for performing a method including the following steps:
forming a contig based on at least one group of sequence data for a selected sample;
displaying the data in reverse and forward sequences;
determining a list of positive probes based on at least one of the reverse and forward sequences;
displaying the list of positive probes;
displaying a chromatogram associated with the sample; and
displaying a database of the sequence data.

32. A computer program product according to claim 31, wherein the method further includes permitting a user to edit the database.

33. A computer program product according to claim 31, wherein the method further includes determining a score for the sequence data and comparing said score with a score obtained by SSO.

34. A computer program product according to claim 31, wherein the method further includes displaying a list of negative probes.

35. A computer program product according to claim 31, wherein the program product is a web-based application.
